(19) United States
(12) Patent Application Publication
(10) Pub. No.: US 2012/0209294 A1
(43) Pub. Date: Aug. 16, 2012

(54) HANDHELD MICRODERMABRASION DEVICE AND METHODS OF USING THE SAME

(75) Inventors: Diane Bitaraf, Orem, UT (US); Buckley Barlow, Holladay, UT (US); Travis Dowdell, Holladay, UT (US)

(73) Assignee: Riiviva, Inc.

(21) Appl. No.: 13/407,567

(22) Filed: Feb. 28, 2012

Related U.S. Application Data
(63) Continuation-in-part of application No. 13/271,979, filed on Oct. 12, 2011.
(60) Provisional application No. 61/392,372, filed on Oct. 12, 2010.

(51) Int. Cl. A61B 17/32

(52) U.S. Cl. 606/131

(57) ABSTRACT

An integrated hand-held device for microdermabrasion includes a device housing configured to be handheld. An abrasive suction tip attached to the device housing allows air to flow into the device housing through a tip opening and a secondary inlet separate from the tip opening. The abrasive suction tip includes an abrasive surface. A vacuum motor enclosed within the device housing provides air flow suction through the abrasive suction tip. An electrical storage component enclosed within the device housing provides electrical power to the vacuum motor. A switch electrically coupled to the vacuum motor and the electrical storage component can power on the vacuum motor.

### RELATED APPLICATIONS

This application is a continuation-in-part of U.S. patent application Ser. No. 13/271,979, filed on Oct. 12, 2011, which claims the benefit of U.S. patent application Ser. No. 61/392,372, filed on Oct. 12, 2010, which are each incorporated by reference.

### FIELD OF THE INVENTION

The present invention relates generally to microdermabrasion devices. Accordingly, the present invention involves the fields of microdermabrasion and skin care.

### BACKGROUND

Microdermabrasion can be used to exfoliate and stimulate the skins' surface. Microdermabrasion can stimulate collagen production in the body, which can result in a supple skin tone and texture. Microdermabrasion can be used to reduce fine lines, crow's feet, age spots, acne scars, blemishes, enlarged pores, blackheads, stretch marks, and sun damage. Typically, microdermabrasion is provided by dermatologists and salon professionals in doctor offices, salons, and spas.

### SUMMARY OF THE INVENTION

An integrated hand-held device for microdermabrasion includes a device housing configured to be held in a hand. An abrasive suction tip attached to the device housing allows air to flow into the device housing through a tip opening and a secondary inlet separate from the tip opening. The abrasive suction tip includes an abrasive surface. A vacuum motor enclosed within the device housing provides air flow suction through the abrasive suction tip. In an example, the vacuum motor can be operated at a single speed while also allowing for variable adjustment of vacuum strength at the suction tip via the tip opening and a secondary inlet. An electrical storage component enclosed within the device housing provides electrical power to the vacuum motor and other components. For example, the electrical storage component can be a rechargeable battery. A switch electrically coupled to the vacuum motor and the electrical storage component can power on the vacuum motor.

In an example, the secondary inlet can include a secondary channel through the abrasive suction tip having a secondary inlet opening at the abrasive surface. The tip opening can have a diameter at least two times a diameter of a secondary inlet opening of the secondary inlet. A diameter of the secondary channel can be at least two times the diameter of the secondary inlet opening. The tip opening can be substantially axially centered in the abrasive suction tip. The secondary inlet can be located within a peripheral area of the abrasive surface distanced from the tip opening. The abrasive surface of the abrasive suction tip can be annular allowing air to flow through the center of the abrasive suction tip and the secondary inlet can be located within the abrasive surface. The abrasive suction tip can include exterior threads oriented circumferentially about the abrasive suction tip configured for attaching attachments to the abrasive suction tip.

In another example, the switch of the integrated hand-held device (i.e., hand-held device or hand-held microdermabrasion device) can include a bypass air port. The switch allows a variable inlet air flow volume to pass through the bypass air port with a change in position of the switch. With the bypass air port, the air flow can allow inlet air into the vacuum motor to come from both the abrasive suction tip and the bypass air port. The variable inlet air flow volume passing through the bypass air port can use exhaust from the vacuum motor, which re-circulates the air passing through the vacuum motor. When the switch is slid or positioned into various positions the suction pressure of the abrasive suction tip can increase or decrease based on the air flow volume passing through the bypass air port.

The switch can be placed between the abrasive suction tip and the vacuum motor. In this manner, the vacuum motor can be operated at a single speed while also allowing for variable adjustment of vacuum strength at the suction tip.

In another configuration, the switch can be operatively connected or adjacent to an air channel from the abrasive suction tip to the vacuum motor. A switch housing of the switch can include the bypass air port. A switch actuator of the switch can include actuator air ports. When the switch actuator moves relative to the switch housing in various positions, the various actuator air ports align with the bypass air port of the switch housing allowing a variable inlet air flow volume to pass through the bypass air port into the vacuum motor. The actuator air ports may be various sizes or a variable number at different sections of the switch actuator allowing variable air flow volume through the bypass air port at different switch positions.

A method is also provided for using an integrated hand-held device for microdermabrasion. A skin surface is provided. An abrasive suction tip on the hand-held device is applied to the skin surface. The hand-held device includes a single-speed vacuum motor, an electrical storage component, and a switch. The single-speed vacuum motor provides air flow suction through the abrasive suction tip. The abrasive suction tip allows air to flow into a device housing through a tip opening and a secondary inlet separate from the tip opening. The electrical storage component provides electrical power to the vacuum motor. The switch is operatively connected between the abrasive suction tip and the vacuum motor. The switch powers on the vacuum motor.

The abrasive suction tip can be moved across the skin surface. The removed skin from the abrasion can be lifted away from the skin surface with the suction pressure of the hand-held device. The abrasive suction tip can be positioned on the skin surface to provide additional suction pressure when covering both the tip opening and the secondary inlet with the skin surface.

In another example, a suction pressure of the abrasive suction tip can be adjusted by changing a position of the switch. A change in the switch position allows a variable inlet air flow volume to through a bypass air port within the switch.

The method can reduce fine lines and treat skin conditions such as crow's feet, age spots, acne scars, blemishes, enlarged pores, blackheads, stretch marks, and sun damage. Furthermore, these methods can reduce the appearance of fine lines, wrinkles, hyperpigmentation, acne scars, sun damage, age spots, stretch marks, blackheads, and enlarged pores, as well as a treatment for keratosis pilaris. It can also stimulate collagen and blood flow, remove dead skin cells, and smooth and soften the skins' texture, and improves overall skin tone.

The integrated hand-held microdermabrasion device provides for salon quality microdermabrasion for home use at a relatively low cost. The integrated hand-held microdermabrasion device is portable and can also be stored easily without occupying significant space. The vacuum motor of the integrated hand-held microdermabrasion device can be a simple and inexpensive single-speed motor that provides for various suction forces or pressures.

There has thus been outlined, rather broadly, the more important features of the disclosure so that the detailed description that follows may be better understood, and so that the present contribution to the art may be better appreciated. Other features of the disclosure will become clearer from the following detailed description of the disclosure, taken with the accompanying drawings and claims, or may be learned by the practice of the disclosure.

### BRIEF DESCRIPTION OF THE DRAWINGS

FIG. 1A is an illustration of a cross-section view of an integrated hand-held device for microdermabrasion in accordance with an example.

FIG. 1B is an illustration of a cross-section view of an abrasive suction tip assembly including a tip opening and a secondary inlet in accordance with an example.

FIG. 2A is an illustration of an annular abrasive suction tip of an integrated hand-held device for microdermabrasion including a tip opening and a secondary inlet opening in accordance with an example.

FIG. 2B is a top view of the annular abrasive suction tip of FIG. 2A.

FIG. 3 is an illustration of a tip adapter of an integrated hand-held device for microdermabrasion in accordance with an example.

FIG. 4A is a side view of a suction bypass switch assembly of an integrated hand-held device for microdermabrasion in accordance with an example.

FIG. 4B is a top perspective view of the suction bypass assembly of FIG. 4A.

FIG. 5 is an illustration of a slide switch bypass housing of an integrated hand-held device for microdermabrasion in accordance with an example.

FIG. 6 is an illustration of a bypass slide switch actuator of an integrated hand-held device for microdermabrasion in accordance with an example.

FIG. 7 is an illustration of a collection canister of an integrated hand-held device for microdermabrasion in accordance with an example.

FIG. 8 is an illustration of a collection canister assembly including the canister and plenum of an integrated hand-held device for microdermabrasion in accordance with an example.

FIG. 9A is a bottom perspective view of a collection plenum of an integrated hand-held device for microdermabrasion in accordance with an example.

FIG. 9B is a top view of the collection plenum shown in FIG. 9A.

FIG. 10 is an illustration of a duct elbow of an integrated hand-held device for microdermabrasion in accordance with an example.

FIG. 11 is a process flow diagram for using an integrated hand-held device for microdermabrasion in accordance with an example.

These drawings merely depict exemplary embodiments of the disclosure, therefore, the drawings are not to be considered limiting of its scope. It will be readily appreciated that the components of the disclosure, as generally described and illustrated in the figures herein, could be arranged, sized, and designed in a wide variety of different configurations. Nonetheless, the disclosure will be described and explained with additional specificity and detail through the use of the accompanying drawings.

### DETAILED DESCRIPTION

It is to be understood that this disclosure is not limited to the particular structures, process steps, or materials disclosed, but is extended to equivalents as would be recognized by those ordinarily skilled in the relevant arts. Alterations and further modifications of the illustrated features, and additional applications of the principles of the examples, which would occur to one skilled in the relevant art and having possession of this disclosure, are to be considered within the scope of the disclosure. It should also be understood that terminology employed herein is used for the purpose of describing particular embodiments only and is not intended to be limiting. The same reference numerals in different drawings represent the same element.

It must be noted that, as used in this specification and the appended claims, the singular forms “a,” “an,” and “the” include plural referents unless the context clearly dictates otherwise. Thus, for example, reference to “an aperture” includes one or more of such openings, reference to “battery” includes reference to one or more of such devices, and reference to “applying” includes one or more of such steps.

In describing and claiming the present disclosure, the following terminology will be used in accordance with the definitions set forth below. As used herein, “substantial” when used in reference to a quantity or amount of a material, or a specific characteristic thereof, refers to an amount that is sufficient to provide an effect that the material or characteristic was intended to provide. Therefore, “substantially free” when used in reference to a quantity or amount of a material, or a specific characteristic thereof, refers to the absence of the material or characteristic, or to the presence of the material or characteristic in an amount that is insufficient to impart a measurable effect, normally imparted by such material or characteristic.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.6 mm to about 0.3 mm” should be interpreted to include not only the explicitly recited values of about 0.6 mm and about 0.3 mm, but also include individual values and sub-ranges within the indicated range. Thus, included in this numerical range are individual values such as 0.4 mm and 0.5, and sub-ranges such as from 0.5-0.4 mm, from 0.4-0.35, etc. This same principle applies to ranges reciting only one numerical value. Furthermore, such an interpretation should apply regardless of the breadth of the range or the characteristics being described.

As used herein, the term “about” means that dimensions, sizes, formulations, parameters, shapes and other quantities and characteristics are not and need not be exact, but may be approximated and/or larger or smaller, as desired, reflecting tolerances, conversion factors, rounding off, measurement error and the like and other factors known to those of skill in the art. Further, unless otherwise stated, the term “about” shall expressly include “exactly,” consistent with the discussion above regarding ranges and numerical data.

In the present disclosure, the term “preferably” or “preferred” is non-exclusive where it is intended to mean “preferably, but not limited to.” Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the disclosure should be determined solely by the appended claims and their legal equivalents, rather than by the descriptions and examples given herein.

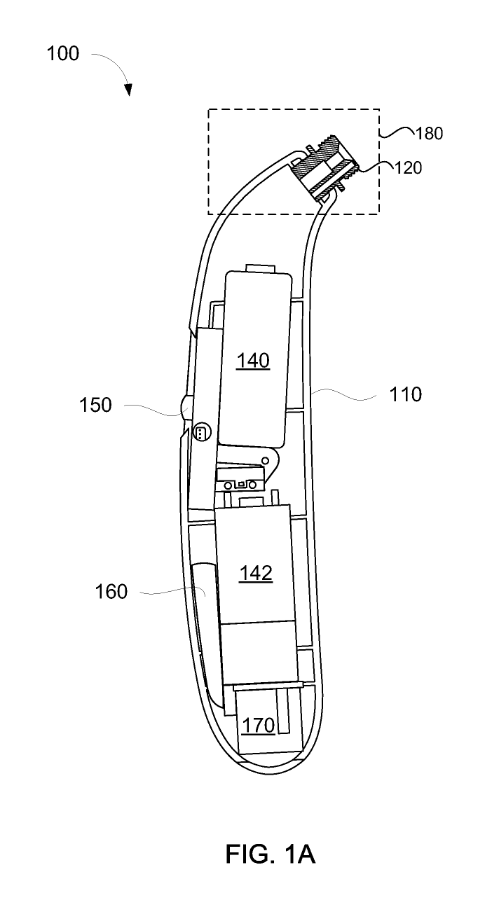

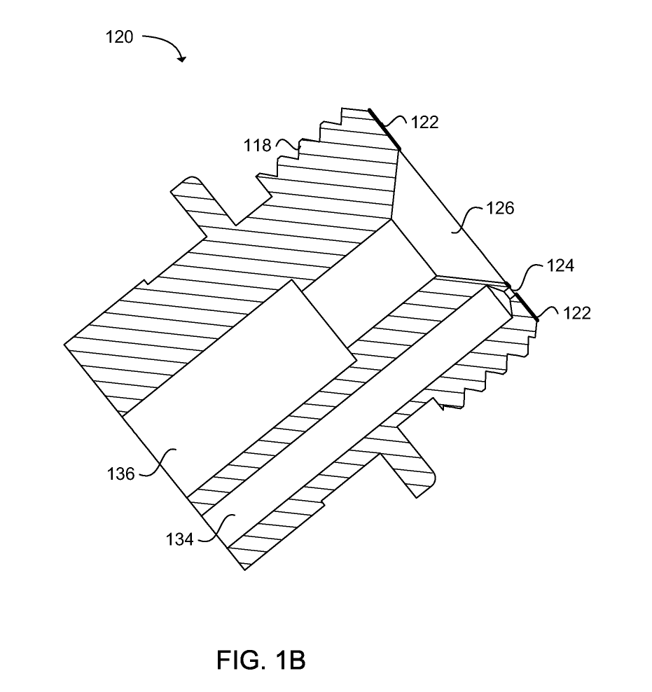

FIG. 1A illustrates an integrated hand-held device 100 for microdermabrasion (MDA). The integrated hand-held device includes a device housing 110 configured to be held in a hand. An abrasive suction tip assembly 120 attached to the device housing allows air to flow into the device housing. A vacuum motor 130 enclosed within the device housing provides air flow suction through the abrasive suction tip. An electrical storage component 140 enclosed within the device housing provides electrical power to the vacuum motor. The electrical storage component can be a rechargeable battery. The rechargeable battery can include lead-acid, nickel cadmium (NiCd), nickel metal hydride (NiMH), lithium ion (Li-ion), lithium ion polymer (Li-ion polymer), or combination of these materials. However, disposable batteries can also be suitable. A switch 150 can be electrically coupled to the vacuum motor 130 and the electrical storage component 140 can power on the vacuum motor. The integrated hand-held device can include a collection canister 170 for collecting skin remnants and other particles sucked into the device housing through the abrasive suction tip. An air channel 160 can direct air from the abrasive suction tip to the vacuum motor and/or collection canister.

FIG. 1B is an exploded view from tip section 180 in FIG. 1A which further illustrates the abrasive suction tip assembly 120 including a tip opening and a secondary inlet. The abrasive suction tip assembly can include an abrasive suction tip and a tip adapter. The abrasive suction tip can be configured to be applied to a treatment surface, such as a skin surface. The abrasive suction tip can include an abrasive surface 122.

The tip adapter can provide an attachment or mounting to the device housing. The abrasive suction tip and the tip adapter can be separate components which can be removably connected together (e.g. threads, detent, or the like). In another example, the abrasive suction tip and the tip adapter can be integrated into a single component, as shown in FIG. 1B. The tip opening can include a primary inlet opening 126 at the abrasive surface 122 and a primary inlet channel 136 for allowing air to flow into the device housing from the primary inlet opening of the tip opening. The secondary inlet can include a secondary channel 134 through the abrasive suction tip allowing air to flow into the device housing from a secondary inlet opening 124 at the abrasive surface 122.

In an example, a diameter of the secondary inlet channel 134 can be at least two times the diameter of the secondary inlet opening 124. In another example, a diameter of the secondary inlet channel can be at least four times the diameter of the secondary inlet opening. In another example, an opening area of the secondary inlet opening in the abrasive suction tip can be at least ten times the cross-sectional area of a section of the secondary inlet channel. In an embodiment, the tip opening can have a diameter at least two times a diameter of a secondary inlet opening of the secondary inlet. In another embodiment, the primary inlet channel 136 of the tip opening can have a diameter at least two times a diameter of a secondary inlet opening of the secondary inlet and a diameter at least one and half times a diameter of a secondary inlet channel of the secondary inlet. In another embodiment, a primary inlet channel of the tip opening can have a diameter at least 5 times a diameter of a secondary inlet opening of the secondary inlet, and a primary inlet opening 126 of the tip opening can have a diameter at least 25 times a diameter of a secondary inlet opening of the secondary inlet. In another embodiment, the tip opening can have an opening area in the abrasive suction tip that is at least ten times the opening area of the secondary inlet opening.

In another example, the primary tip opening can be substantially axially centered in the abrasive suction tip and the secondary inlet can be located within a peripheral area of the abrasive surface 122 distanced from the primary tip opening 126. In another example, the abrasive surface of the abrasive suction tip can be annular, allowing air to flow through the center of the abrasive suction tip (or tip opening) and the secondary inlet can be located within the abrasive surface. The tip opening and/or the secondary inlet can be circular, rectangular, triangular, other type of polygon, or any other suitable geometry. In an example, the primary inlet opening of the tip opening can have a tapered, cone, or funnel shape (or trapezoidal cross-sectional area), as shown in FIG. 1B. The funnel shape can generate a vortex air flow and/or channel air flow and debris into the device housing. The primary inlet channel can have a cylindrical or cuboidal shape (or rectangular cross-sectional area).

The primary tip opening can be fluidly connected to the vacuum motor. For example, a channel or tube can be provided to connect the primary tip to the vacuum motor. As such, the secondary opening can open into an interior of the housing. Covering the tip opening with an application surface (e.g., a skin surface) can provide a specified suction pressure (or an air vortex) on the surface. The vacuum motor can generate a first sound wave or first sound while generating the specified suction pressure. The suction pressure can vary based on a physical force or pressure applied by the abrasive suction tip on the application surface. Covering the secondary inlet opening along with the tip opening can provide additional suction pressure (or different air vortex) on the application surface greater than the specified suction pressure (or the air vortex). The vacuum motor can generate a second sound wave or second sound when the openings are covered which is distinct from a first sound wave or first sound when unobstructed, while generating the additional suction pressure. The second sound wave can generally have a higher frequency or a higher pitch than the first sound wave. The sound waves can provide feedback to a user of a relative suction pressure applied to an application surface by the integrated hand-held device. This audible feedback can also be useful to gauge functioning of the device. The additional suction pressure can provide increased removal of a surface material (e.g., skin particles) than the specified suction pressure achieved using a single primary opening and no secondary opening. A majority of removed surface material can flow through the tip opening. The secondary inlet can provide a variable suction pressure, when the vacuum motor is a single-speed motor. In another example, the abrasive suction tip can include a plurality of secondary inlets, where each inlet can alter the suction pressure of the abrasive suction tip. Such additional secondary inlets can be distributed across the abrasive surface or along peripheral edges of the tip assembly, including outer facing edges.

In another aspect, the abrasive suction tip assembly 120 can include exterior threads 118 oriented circumferentially about a circular abrasive suction tip configured for attaching attachments to the abrasive suction tip. The exterior threads may provide a male threaded tip connection to a female threaded attachment. Different attachments can be used for treating different types of skin conditions.

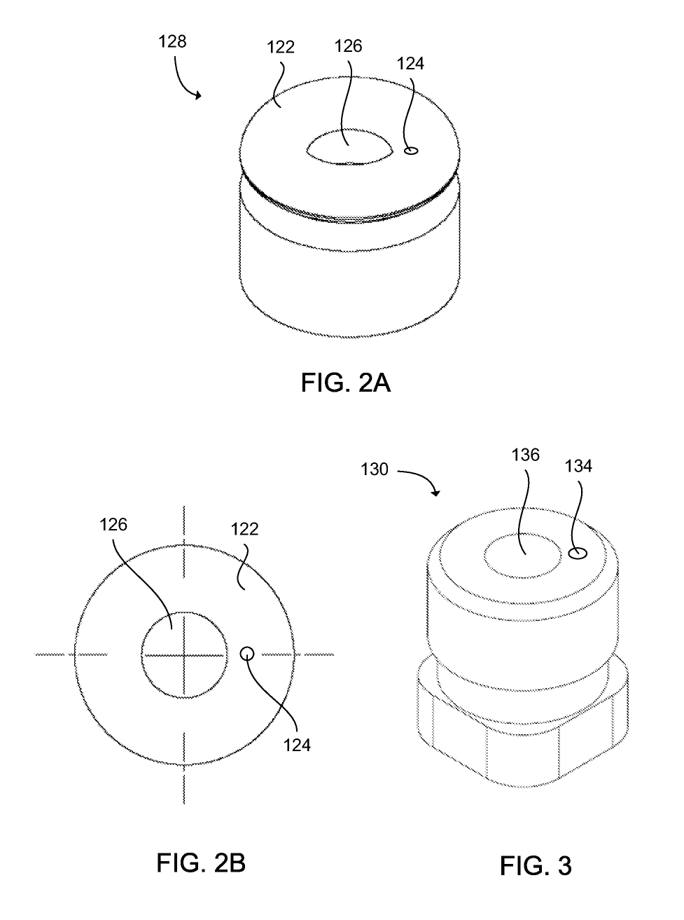

FIG. 2A illustrates an example of an abrasive suction tip 128. The abrasive suction tip can have an abrasive surface 122. The abrasive surface can have an annular shape as shown (e.g., donut shape) allowing air flow through the center tip opening 126, although other shapes can be suitable. The abrasive surface can have a contoured arc shape allowing air flow adjacent to the arc. The abrasive surface can form various types of shapes and surfaces that allow for suction near the abrasive surface and air flow into the device housing. Non-limiting examples of alternative abrasive surfaces can include arcuate, concentric rings, blocks, circular, etc. In each case, the contact surface can be rounded near edges to provide comfort to the user and avoid potential skin damage. The abrasive suction tip can include a secondary inlet opening 124 of the secondary inlet. As shown generally in FIG. 2B, in one example, an outside diameter of the abrasive suction tip can have a diameter between 0.2 inches and 2 inches. In another example, the outside diameter of the abrasive suction tip has a diameter between 0.3 inches and 0.7 inches. The inside diameter of an opening 126 within the abrasive suction tip can be proportional to and less than the outside diameter of the abrasive suction tip. In another example, an inside diameter of the tip opening or an outside diameter of the abrasive suction tip can have a diameter greater than a device housing diameter where the device housing is configured to be held in the hand.

The abrasive surface 122 can be formed from suitable microdermabrasive materials. For example, ablations on a solid material can be used for the tip and can form the abrasive surface. In one example, the abrasive suction tip or an abrasive surface can be formed of stainless steel. Suitable stainless steel can be a steel alloy with a minimum of 10.5% chromium content by mass. Stainless steel can include, but is not limited to, American Iron and Steel Institute (AISI) Types 201, 202, 301, 304, 304L, 304H, 309S, 310S, 316, 316L, 316I, 316Ti, 321, 321H, 347, 347H, and 430. The stainless steel can be a medical grade stainless steel. Medical grade stainless steel can include AISI type 316, 316L (UNS S31603), or 3161. Type 316L stainless steel can include the following chemical components: iron (Fe), <0.03% carbon (C), 16-18.5% chromium (Cr), 10-14% nickel (Ni), 2-3% molybdenum (Mo), <2% manganese (Mn), <1% silicon (Si), <0.045% phosphorus (P), and <0.03% sulfur (S). In another example, type 316L stainless steel can include approximately (in weight percentage) 0.03% carbon (C), 2.00% manganese (Mn), 1.00% silicon (Si), 16.0-18.0% chromium (Cr), 10.0-14.0% nickel (Ni), 0.045% phosphorus (P), 0.03% sulfur (S), 2.0-3.0% molybdenum (Mo), and the base metal iron (Fe). In another example, the abrasive surface can include bonded diamonds. Other non-limiting examples of suitable abrasives can include alumina (i.e. aluminum oxide crystals), micronized mineral crystals, corundum powder, cubic boron nitride, zirconia, tungsten, and the like. These abrasive particulates can be bonded to a tip substrate using any approach which secures the abrasives to prevent fallout during extended use. Non-limiting examples of suitable bonding mechanisms can include curable adhesives, resins, electroplating, brazing, chemical bonding, carbide bonding, and the like.

In another example, the abrasive suction tip can coated with various secondary agents. Alternatively, secondary agents can be provided as a separate post-treatment option. Non-limiting examples of suitable secondary agents can include an organic mineral, a serum (e.g. vitamin A, vitamin D, vitamin E, etc.), a chemical (e.g. glycolic acid, resorcinol, lactic acid, etc.), or combinations these materials. The serum can include organic compounds or a biological organic serum. Organic compounds can be compounds derived from plants where synthetic pesticides, chemical fertilizers, sewage sludge, or genetically modified organisms (GMOs) were not used. The biological organic serum can include secretions by an animal, such as land snails, or an extract from a plant, such as salicylic acid in willow bark extract. The biological organic serum can include biological activators that can enhance the human skin's own growth factors and can trigger the reproduction of brand new skin cells. The biological organic serum can include contains enzymes that digest keratin, where an excess of keratin can generate skin conditions, such as keratosis pilaris. The organic mineral can include an oxalate, a mellitate, a citrate, a cyanate, an acetate, a formate, a mineral oil, or combinations these materials. In another example, the organic mineral can include a whewellite, a moolooite, a mellite, a fichtelite, a carpathite, an evenkite, an abelsonite, humic acid, fulvic acid, or combinations these materials. Organic minerals can include biogenic substances in which geological processes have been a part of the genesis or origin of the existing compound. A biogenic substance can be a substance produced by life processes, such as constituents or secretions of plants or animals. Organic compounds, biological organic serums, and organic minerals can have properties that are non-toxic to living organisms.

Referring again to FIG. 1A, the abrasive suction tip assembly 120 can be in a fixed position relative to the device housing. Alternatively, the abrasive suction tip can be configured to tilt relative to the device housing allowing for the abrasive surface 122 to be placed flat on a treatment surface (e.g., a skin surface) and contour to the treatment surface even when the device is not properly oriented on the treatment surface or when moved over various contours of the body. Often, the device may be positioned incorrectly by the way the user holds the device relative to the treatment surface. Allowing the tip to tilt provides for additional comfort and less rigidity in the positioning of the housing relative to a skin surface. Non-limiting examples of mechanisms which allow for tilt can include spring levers, multi-axis pivots, and the like. The abrasive surface of abrasive suction tip can tilt within about 60° of rotation from the axis of attachment to the device housing although other tilts can be provided. In one alternative, the abrasive surface can tilt in all directions (e.g., 360°) of the surface allowing the device to contour in all directions to the treatment surface.

The abrasive suction tip can be removable from the device housing allowing the abrasive suction tip to be cleaned, replaced, or changed with a tip with a different type or grade of abrasive surface. The abrasive suction tip can be replaceable and detachable. In an example, the replaceable and/or detachable abrasive suction tip can attach to a tip adapter. The abrasive suction tip assembly can include the tip adapter 130, as illustrated in FIG. 3 which can facilitate replacement of the abrasive suction tip. The tip adapter can include a primary inlet channel 136 of the tip opening and a secondary inlet channel 134 of the secondary inlet to allow air to flow into the device housing from the abrasive suction tip.

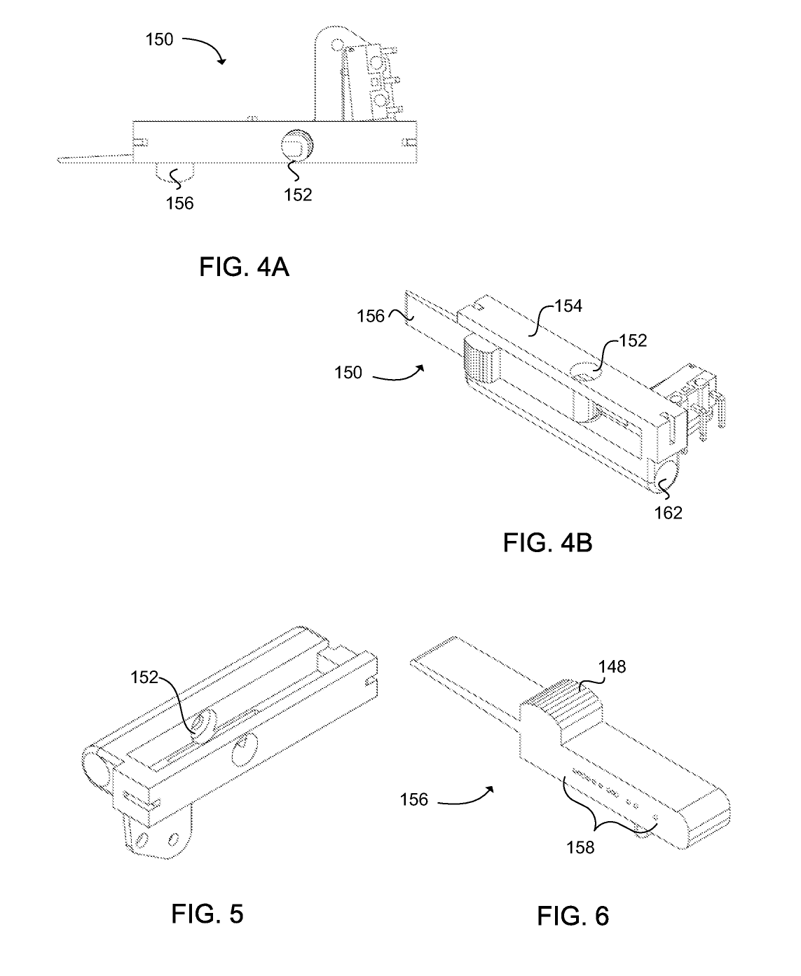

In another example, the switch 150 of the integrated hand-held device (i.e., hand-held device or hand-held microdermabrasion device) can include a bypass air port 152, as illustrated in FIGS. 4A, 4B and 5. The switch allows a variable inlet air flow volume to pass through the bypass air port with a change in position of a switch actuator 156. With the bypass air port, the air flow can pass through both the abrasive suction tip and the bypass air port before reaching the vacuum motor. The variable inlet air flow volume passing through the bypass air port can use exhaust from the vacuum motor, which re-circulates the air passing through the vacuum motor. The variable inlet air flow volume passing through the bypass air port can use air pulled into the switch from outside the device and optionally exclude air already passing through abrasive suction tip. When the switch is slid or positioned into various positions the suction pressure of the abrasive suction tip can increase or decrease based on the air flow volume passing through the bypass air port. The switch can be located between the abrasive suction tip and the vacuum motor.

The switch 150 can be operatively connected or adjacent to an air channel from the abrasive suction tip to the vacuum motor. A switch housing of the switch can include the bypass air port 152 and a portion of the air channel 162. A switch actuator 156 of the switch can include actuator air ports 158 and slide within the switch housing, as illustrated in FIGS. 4A, 4B and 6. When the switch actuator moves relative to the switch housing in various positions, the actuator air ports 158 can align with the bypass air port 152 of the switch housing allowing a variable inlet air flow volume to pass through the bypass air port into the vacuum motor. The actuator air ports may be various sizes or a variable number at different sections of the switch actuator allowing variable air flow volume through the bypass air port. Thus, the number and/or area of the aperture or apertures which are aligned with the bypass air port determine the proportion of inlet air into the vacuum motor which is pulled from the suction tip versus from open areas within the housing. As such, the effective vacuum strength at the suction tip 120 varies depending on the switch actuator position and bypass air volume. The switch actuator can include a thumb grip 148 to allow a user to move the switch actuator into different positions. The switch can be indexed or continuously slide from one position to another. The switch can both power on the hand-held microdermabrasion device and control the variable air flow volume through the bypass air port into the air channel.

For example, the switch 150 can have various settings including: off, low, medium, and high. The off setting disconnects the vacuum motor from the electrical storage component. The low setting powers the vacuum motor and allows the greatest air flow volume through the bypass air port 152. The bypass air port or the actuator air ports for the low setting may be the largest size or the greatest number relative to the other switch settings. The high setting can power the vacuum motor and block the bypass air port preventing inlet air flow through the bypass air port. The medium setting powers the vacuum motor and allows some air flow volume through the bypass air port. The switch can have various settings, continuously variable, or no settings between the high and the low settings.

The switch housing 154 can have a single bypass air port 152 that allows the variable inlet air flow volume into an air channel. The switch actuator 156 can have multiple actuator air ports 158 that allow variable inlet air flow volume to be passed through the switch when each of the multiple actuator air ports aligns with the single bypass air port. These actuator air ports can be multiple holes (as shown) or any other suitable opening shape (i.e. slits, ovals, tapered slot, or the like).

In another configuration not shown, the switch actuator can have a single actuator air port that allows the variable inlet air flow volume into an air channel. The switch housing can have multiple bypass air ports that allow variable inlet air flow volume to be passed through the switch when the single actuator air port aligns with each of the multiple bypass air ports.

In another example, the device housing includes an ultrasonic emitter (not shown) for emitting ultrasonic waves adjacent to the abrasive suction tip. Ultrasound is cyclic sound pressure with a frequency greater than the upper limit of human hearing (20 kilohertz (kHz)).

The emitted ultrasonic waves can have a frequency between 100 kHz and 50 megahertz (MHz). The ultrasonic waves can be used to assist in loosening or removing a surface material, such as skin cells and debris. In one alternative, the ultrasonic emitter can be an attachment that can be threaded on to the abrasive suction tip. Optionally, the ultrasonic emitter can be integrated into the device adjacent the tip and oriented to direct ultrasound toward the abrasive surface and/or treatment surface.

The vacuum motor enclosed in the device housing can be a single-speed motor. A single-speed motor can be more inexpensive than a variable-speed motor for the same power levels and suction forces and pressures. A single-speed motor assembly may also be more reliable than a variable-speed motor assembly and use fewer components and less expensive components than the variable-speed motor assembly. Using the bypass port with the single-speed motor can allow for finer adjustments in suction pressure or force than the variable-speed motor can provide. Alternatively, the vacuum motor can be a variable speed motor. Although not required, a variable speed motor can allow for elimination of the bypass switch as described above.

The electrical storage component can be a self-contained power source, like an electrochemical device (e.g., a battery) or an electric device (e.g., capacitor, super-capacitor, or ultra-capacitor, inductor, solar cell, or similar electrical device that can store energy). The electrical storage component can be recharged by an electrical connection to commercial alternating current (AC) power outlet through a power cord. The device housing may include an electrical jack or plug to provide an electrical connection to recharge the electrical storage component. Alternatively, the device housing can include a removable cover to allow for replacement or recharging of batteries.

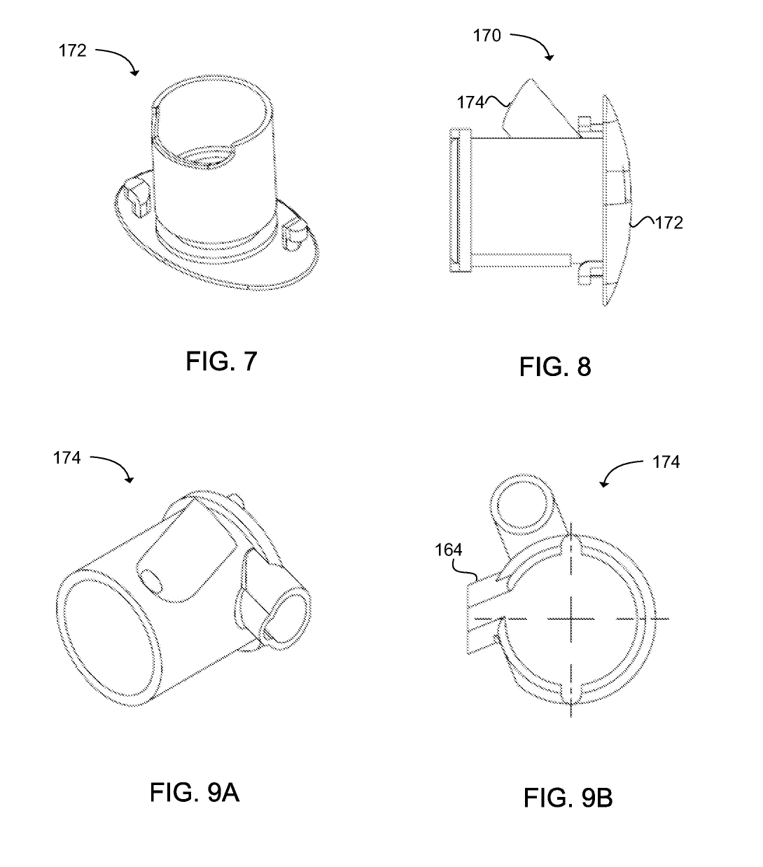

During operation of the device, skin cells are removed from the treatment surface along with other debris. These skin cells and debris can be optionally filtered to prevent recirculation and blockage of the vacuum motor and/or other passages within the device housing. The hand-held microdermabrasion device can include a filter for filtering the air of skin and debris before entering the vacuum motor. A collection canister 172 for collecting and holding the skin and debris generated from the hand-held device during a microdermabrasion treatment, as illustrated in FIGS. 7 and 8. The filter and the collection canister can be operatively connected between the abrasive suction tip and the vacuum motor. The filter can be included within the collection canister or can be a component removed separately from the collection canister. The filter can generally be a foam or fibrous material, although other filter media can be used. The filter or collection canister can be replaceable and removable from the device housing to facilitate cleaning and/or replacement. The collection canister of a collection canister assembly 170 can be mated with a collection plenum 174 within the device housing, as illustrated in FIG. 9A and 9B. The collection plenum provides a chamber in which air can gather prior to entering the collection canister.

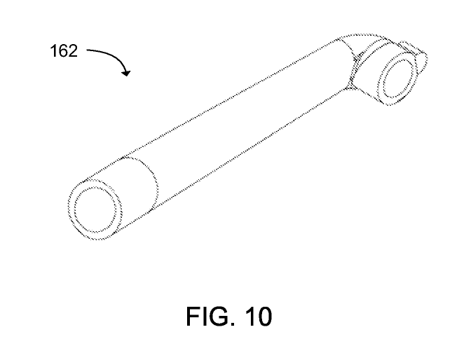

The air channel can be formed by the device housing. The air channel can optionally include tubing for sections of the air channel. The air channel can provide an air path from the abrasive suction tip to the vacuum motor. The air channel can provide a path for the skin particles and debris included in the air flow. The air channel can be adjacent to the switch and the bypass air port of the switch. The air channel can include an air path from the vacuum motor air exhaust to the switch bypass air port to allow for re-circulation of air of the air channel and vacuum motor. The air channel can include the collection plenum 174 which can be connected to a duct elbow 162 via plenum inlet 164, as illustrated in FIG. 9B and 10.

The integrated hand-held device can be used for microdermabrasion treatments. A method is provided for using an integrated hand-held device for microdermabrasion. A skin surface with a skin condition is provided. An abrasive suction tip on the hand-held device is applied to the skin surface. The hand-held device includes a single-speed vacuum motor, an electrical storage component, and a switch. The single-speed vacuum motor provides air flow suction through the abrasive suction tip via the tip opening and the secondary inlet. The electrical storage component provides electrical power to the vacuum motor. The switch is operatively connected between the abrasive suction tip and the vacuum motor. The switch powers on the vacuum motor. In an example, the suction pressure of the abrasive suction tip can be adjusted by changing a position and/or the force of the abrasive suction tip on the application surface where the tip opening, the secondary inlet, or both the tip opening and the secondary inlet can be covered by the application surface. The abrasive suction tip can be moved across the skin surface. The removed skin from the abrasion can be sucked from the skin surface with the suction pressure of the hand-held device.

In another example, the suction pressure of the abrasive suction tip can be adjusted by changing a position of the switch. A change in the position allows a variable inlet air flow volume to through a bypass air port within the switch.

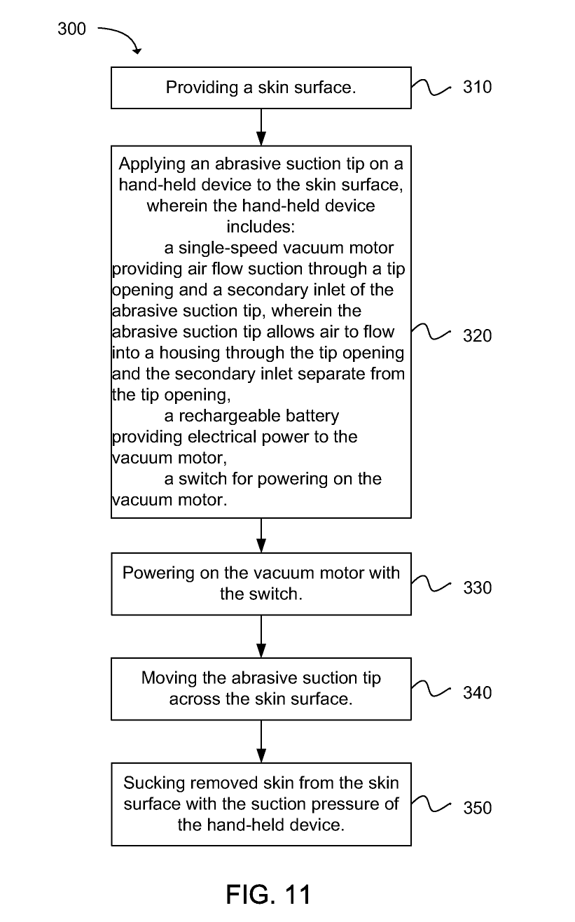

Another example provides a method 300 of using an integrated hand-held device for microdermabrasion, as shown in the flow chart in FIG. 11. The method includes the operation of providing a skin surface 310. The operation of applying an abrasive suction tip on a hand-held device to the skin surface 320 follows, wherein the hand-held device includes: a single-speed vacuum motor providing air flow suction through a tip opening and a secondary inlet of the abrasive suction tip, wherein the abrasive suction tip allows air to flow into a housing through the tip opening and the secondary inlet separate from the tip opening; a rechargeable battery providing electrical power to the vacuum motor; and a switch for powering on the vacuum motor. The next operation of the method can be powering on the vacuum motor with the switch 330. The abrasive suction tip can be moved across the skin surface 340. The method can further include sucking removed skin from the skin surface with the suction pressure of the hand-held device 350.

In another example, the method can further include positioning the abrasive suction tip to the skin surface to provide additional suction pressure by covering both the tip opening and the secondary inlet with the skin surface.

The method can reduce fine lines and treat skin conditions such as crow's feet, age spots, acne scars, blemishes, enlarged pores, blackheads, stretch marks, and sun damage. The method can treat forms of keratosis pilaris (KP, also known as follicular keratosis). KP can be a common, autosomal dominant, genetic follicular condition that is manifested by the appearance of rough bumps on the skin, which can occur when the human body produces excess keratin, a natural protein in the skin. The method can reduce the appearance of fine lines, wrinkles, hyperpigmentation, acne scars, sun damage, age spots, stretch marks, blackheads, and enlarged pores. It can stimulate collagen and blood flow, remove dead skin cells, smooth and soften the skins' texture, and improve overall skin tone.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts described. Accordingly, it is not intended that the invention be limited, except as by the claims set forth below.

What is claimed is:

1. An integrated hand-held device for microdermabrasion, comprising:
a device housing configured to be held in a hand;
an abrasive suction tip attached to the device housing allowing air to flow into the device housing through a tip opening and a secondary inlet separate from the tip opening, wherein the abrasive suction tip includes an abrasive surface;
a vacuum motor enclosed within the device housing providing air flow suction through the abrasive suction tip;
an electrical storage component enclosed within the device housing providing electrical power to the vacuum motor;
a switch electrically coupled to the vacuum motor and the electrical storage component for powering on the vacuum motor.

2. The integrated hand-held device of claim 1, wherein the tip opening has a diameter at least two times a diameter of a secondary inlet opening of the secondary inlet.

3. The integrated hand-held device of claim 1, wherein the tip opening is substantially axially centered in the abrasive suction tip and the secondary inlet is located within a peripheral area of the abrasive surface distanced from the tip opening.

4. The integrated hand-held device of claim 1, wherein the abrasive surface of the abrasive suction tip is annular allowing air to flow through the center of the abrasive suction tip and the secondary inlet is located within the abrasive surface.

5. The integrated hand-held device of claim 1, wherein the abrasive suction tip includes exterior threads oriented circumferentially about the abrasive suction tip configured for attaching attachments to the abrasive suction tip.

6. The integrated hand-held device of claim 1, wherein the electrical storage component is a rechargeable battery.

7. The integrated hand-held device of claim 1, wherein the abrasive suction tip comprises stainless steel.

8. The integrated hand-held device of claim 1, wherein the abrasive suction tip is removable from the device housing.

9. The integrated hand-held device of claim 1, wherein the device housing includes an ultrasonic emitter for emitting ultrasonic waves adjacent to the abrasive suction tip.

10. The integrated hand-held device of claim 1, wherein the abrasive suction tip is coated with a material selected from the group consisting of an organic mineral, a serum, a chemical, and combinations thereof.

11. The integrated hand-held device of claim 1, wherein the abrasive suction tip is coated with an organic mineral selected from the group consisting of an oxalate, a mellitate, a citrate, a cyanate, an acetate, a formate, a hydrocarbon, whewellite, a moolooite, a mellite, a fichtelite, a carpathite, an evenkite, an abelsonite, humic acid, fulvic acid, and combinations thereof.

12. The integrated hand-held device of claim 1, wherein the switch includes a bypass air port that is operatively connected between the abrasive suction tip and the vacuum motor, and the switch allows a variable inlet air flow volume through the bypass air port with a change in position of the switch.

13. The integrated hand-held device of claim 1, wherein the switch is slidably connected to a switch housing with a bypass air port, and the bypass air port is operatively connected to an air channel from the abrasive suction tip to the vacuum motor, and the switch includes a plurality of actuator air ports within the switch that allows a variable inlet air flow volume through the bypass air port with a change in position of the switch.

14. The integrated hand-held device of claim 1, wherein the vacuum motor is a single-speed motor.

15. The integrated hand-held device of claim 1, further comprising a removable collection canister operatively connected between the abrasive suction tip and the vacuum motor, and further including an optional filter within the collection canister.

16. The integrated hand-held device of claim 1, wherein the abrasive suction tip is tiltable on the device housing within 60° of rotation.

17. The integrated hand-held device of claim 1, wherein the abrasive surface includes bonded abrasive particles.

18. A method of using an integrated hand-held device for microdermabrasion, comprising:
providing a skin surface;
applying an abrasive suction tip on a hand-held device to the skin surface, wherein the hand-held device includes:
a single-speed vacuum motor providing air flow suction through a tip opening and a secondary inlet of the abrasive suction tip, wherein the abrasive suction tip allows air to flow into a housing through the tip opening and the secondary inlet separate from the tip opening,
a rechargeable battery providing electrical power to the vacuum motor,
a switch for powering on the vacuum motor;
powering on the vacuum motor with the switch;
moving the abrasive suction tip across the skin surface; and
sucking removed skin from the skin surface with the suction pressure of the hand-held device.

19. The method of claim 18, further comprising:
positioning the abrasive suction tip to the skin surface to provide additional suction pressure by covering both the tip opening and the secondary inlet with the skin surface.

20. The method of claim 18, wherein moving the abrasive suction tip and sucking mitigates at least one of fine lines, crow's feet, age spots, acne scars, blemishes, enlarged pores, blackheads, stretch marks, sun damage and keratosis pilaris.
